(19) United States
(12) Patent Application Publication
(10) Pub. No.: US 2025/0316356 A1
(43) Pub. Date: Oct. 9, 2025

(54) INFORMATION PROCESSING DEVICE, INFORMATION PROCESSING METHOD, AND NON-TRANSITORY MACHINE-READABLE STORAGE MEDIUM

(71) Applicant: Hitachi, Ltd., Tokyo (JP)

(72) Inventors: Saiko KON, Tokyo (JP); Sachiho TAKAHASHI, Tokyo (JP); Zisheng LI, Tokyo (JP); Masahiro OGINO, Tokyo (JP); Shuntaro YUI, Tokyo (JP)

(21) Appl. No.: 19/088,331

(22) Filed: Mar. 24, 2025

(30) Foreign Application Priority Data
Apr. 9, 2024 (JP) 2024-062546

(51) Int. Cl. G16H 20/10

(52) U.S. Cl. CPC G16H 20/10

(57) ABSTRACT

An information processing device, comprising: an input unit configured to receive pieces of related information pertaining respectively to a plurality of phenomena; a storage unit configured to store a learning model; and an output unit configured to output the inference result, an arithmetic device configured to: calculate an inner product of a first output value of a hidden layer of the learning model to which a first phenomenon out of the plurality of phenomena is input and a second output value of a hidden layer of the learning model to which a second phenomenon out of the plurality of phenomena is input; and execute deep learning of the learning model based on the pieces of related information pertaining to the plurality of phenomena in a manner that decreases, for each combination of phenomena, a difference between the calculated inner product and ground truth data out of the plurality of phenomena.

### CLAIM OF PRIORITY

The present application claims priority from Japanese patent application JP 2024-62546 filed on Apr. 9, 2024, the content of which is hereby incorporated by reference into this application.

### BACKGROUND OF THE INVENTION

This disclosure relates to an information processing device which trains a drug efficacy inference model.

In drug development, development of a new curative drug requires a huge amount of time and cost. Accordingly, an approach that uses AI to infer drug efficacy is attracting attention. Drug efficacy inference using AI provides a way to check usefulness to a cohort of interest. Drug efficacy inference also requires enormous and complicated data processing. For example, in Y-Ching Tang., “Explainable Drug Sensitivity Prediction through Cancer Pathway Enrichment,” Scientific Reports, 11:3128 (2021), there is disclosed a method of presenting a feature amount that contributes greatly to prediction, with use of Integrated Gradients or a similar method.

Generally speaking, when a drug efficacy prediction AI is applied in a clinical setting such as diagnosis, determination of a treatment course, or drug development, it is required to enable a medical doctor or other specialists to determine validity of a result, and a high level of interpretability is accordingly demanded of the AI. With the technology as described in Y-Ching Tang., “Explainable Drug Sensitivity Prediction through Cancer Pathway Enrichment,” Scientific Reports, 11:3128 (2021), drug efficacy is inferred from feature amounts of various types related to abnormality about a specimen, a structure of a pharmaceutical agent, and mechanism of drug efficacy. Further, integrated gradients are calculated, and, for each of the feature amounts, how and to what degree the feature amount contributes to prediction of treatment effectiveness such as an action that increases the treatment effectiveness or an action that decreases the treatment effectiveness can be checked. However, even when an index for explainability such as integrated gradients is calculated for the feature amounts of various types, how the feature amount contributes to prediction varies and it is accordingly difficult to determine the validity by integrating interpretations of those. Consequently, the technology has been unsuccessful in providing a drug efficacy inference model that is highly reliable.

An object of this invention is to provide a drug efficacy inference model that is highly reliable.

### SUMMARY OF THE INVENTION

The representative one of inventions disclosed in this application is outlined as follows. There is provided an information processing device, comprising: an arithmetic device configured to execute predetermined processing: an input unit configured to receive, as input, pieces of related information pertaining respectively to a plurality of phenomena; a storage unit configured to store a learning model which derives an inference result of the pieces of related information pertaining to the plurality of phenomena; and an output unit configured to output the inference result, wherein the arithmetic device is configured to: calculate an inner product of a first output value of a hidden layer of the learning model to which a first phenomenon out of the plurality of phenomena is input and a second output value of a hidden layer of the learning model to which a second phenomenon out of the plurality of phenomena is input; and execute deep learning of the learning model based on the pieces of related information pertaining to the plurality of phenomena in a manner that decreases, for each combination of phenomena, a difference between the calculated inner product and ground truth data out of the plurality of phenomena.

According to at least one aspect of this invention, a drug efficacy inference model that is highly reliable can be provided. Objects, configurations, and effects other than those described above are clarified in the following description of embodiments.

### BRIEF DESCRIPTION OF THE DRAWINGS

FIG. 1 is a diagram for illustrating a hardware configuration of the information processing device of the first embodiment.

FIG. 2A is a table for showing a configuration example of the specimen data that is included in the learning data as well as the test data in the first embodiment.

FIG. 2B is a table for showing a configuration example of the pharmaceutical agent data that is included in the learning data as well as the test data in the first embodiment.

FIG. 3 is a diagram for illustrating an example of a setting screen output by the information processing device of the first embodiment.

FIG. 4 is a diagram for illustrating an example of an output screen output by the information processing device of the first embodiment.

FIG. 5A is a flow chart of learning processing that is executed by the information processing device of the first embodiment.

FIG. 5B is a flow chart of test processing that is executed by the information processing device of the first embodiment.

FIG. 6 is a diagram for illustrating the learning model in the first embodiment.

FIG. 7 is a diagram for illustrating a hardware configuration of the information processing device of the second embodiment.

FIG. 8 is a diagram for illustrating an example of a setting screen output by the information processing device of the second embodiment.

FIG. 9 is a diagram for illustrating an example of the output screen output by the information processing device of the second embodiment.

FIG. 10 is a flow chart of test processing that is executed by the information processing device of the second embodiment.

FIG. 11 is a diagram for illustrating a hardware configuration of the information processing device of the third embodiment.

FIG. 12 is a table for showing a configuration example of the pathway data in the third embodiment.

FIG. 13 is a diagram for illustrating an example of a setting screen output by the information processing device of the third embodiment.

FIG. 14 is a diagram for illustrating an example of an output screen output by the information processing device of the third embodiment.

FIG. 15 is a flow chart of test processing that is executed by the information processing device of the third embodiment.

### DETAILED DESCRIPTION OF THE PREFERRED EMBODIMENTS

Now, an information processing device 100 according to preferred embodiments of this invention is described with reference to the accompanying drawings. Components having substantially the same functions and configurations are denoted by the same reference symbols in the following description and the accompanying drawings, and an overlapping description thereof is herein omitted.

### First Embodiment

In a first embodiment of this invention, drug efficacy inference is described. Specifically, the information processing device 100 of the first embodiment trains a model in which an inner product of output values of hidden layers learned by deep learning from specimen data 200 and pharmaceutical agent data 203 is used as a drug efficacy score. The information processing device 100 of the first embodiment functions as a treatment effectiveness inference device which outputs drug efficacy information of a selected specimen as a drug efficacy inference result to an output screen 400. The information processing device 100 enables a specialist who is a user, such as a medical doctor, to infer drug efficacy and check usefulness of a pharmaceutical agent to a cohort of interest.

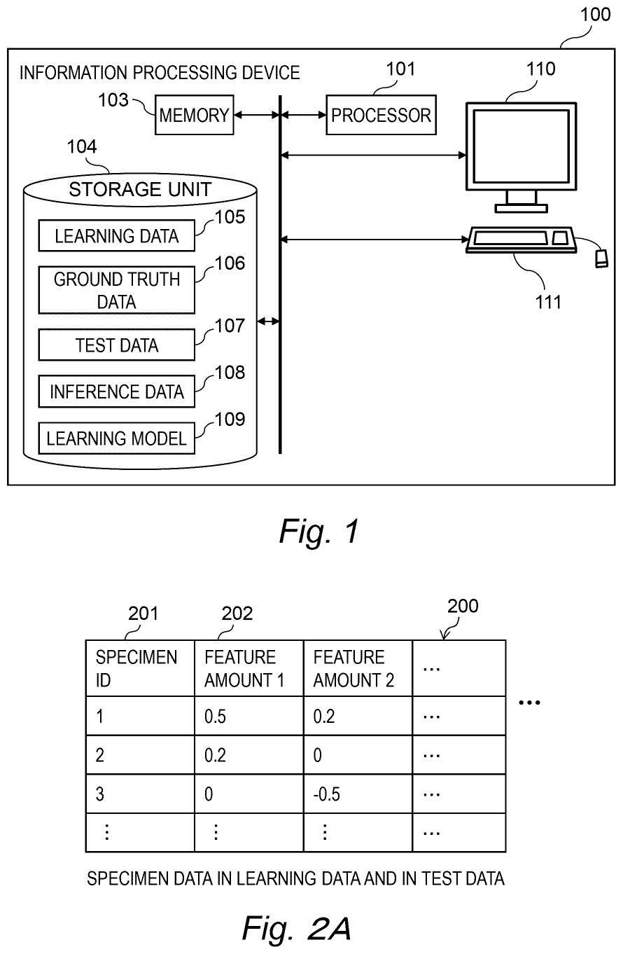

FIG. 1 is a diagram for illustrating a hardware configuration of the information processing device 100 of the first embodiment.

The information processing device 100 is configured from a computer including a processor 101, a memory 103, a storage unit 104, an output unit 110, and an input unit 111.

The processor 101 is an arithmetic device that implements functions of the information processing device 100 by executing a program loaded on the memory 103. As the processor 101, for example, a central processing unit (CPU) or a graphics processing unit (GPU) is usable. The number of processors each used as the processor 101 is not limited to one, and a configuration in which the functions of the information processing device 100 are implemented by a plurality of processors may be employed. Part of processing executed by the processor 101 by running the program may be executed by an arithmetic device of a different format (for example, hardware such as an ASIC or an FPGA).

The memory 103 includes a ROM, which is a non-volatile storage element, and a RAM, which is a volatile storage element. The ROM is a storage device which stores an unchanging program (for example, BIOS) among others. The RAM is a dynamic random access memory (DRAM) or a similar high-speed and volatile storage element, and temporarily stores a program executed by the processor 101 and data used when the program is executed. The storage device is coupled to the arithmetic device.

The storage unit 104 is configured from a storage apparatus that provides a large-capacity and non-volatile storage area, for example, a magnetic storage apparatus (HDD) or a flash memory (SSD). The storage unit 104 stores the data (learning data 105, ground truth data 106, test data 107, inference data 108, and a learning model 109) used by the processor 101 when executing the program, and the program executed by the processor 101. Specifically, the program is read out of the storage unit 104, loaded onto the memory 103, and is executed by the processor 101 to implement the functions of the information processing device 100.

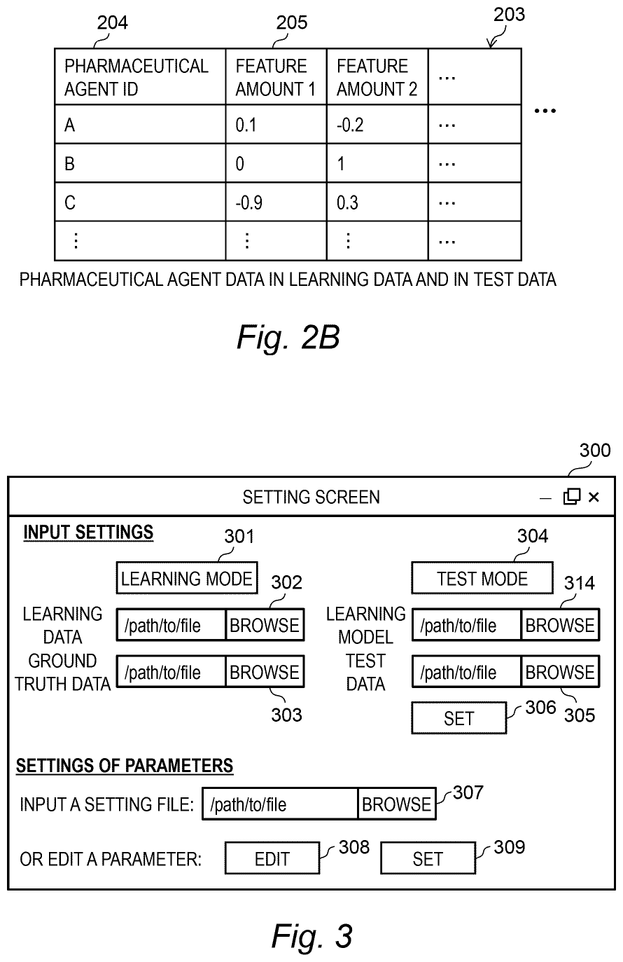

The learning data 105 is data for the learning model 109 to learn through machine learning, or is data already learned by the learning model 109, and includes the specimen data 200 and the pharmaceutical agent data 203. A configuration example of the specimen data 200 is described with reference to FIG. 2A, and a configuration example of the pharmaceutical agent data 203 is described with reference to FIG. 2B. The ground truth data 106 is data that is a ground truth in the specific pair of a specimen and a pharmaceutical agent which exists in the learning data 105, and is associated by the common ID with the learning data 105. Values measured in situations of the learning data 105 are recommended to be used as the ground truth data 106. An example thereof is treatment effectiveness expressed as a drug efficacy score that is associated with the specimen data 200 by a specimen ID 201 and associated with the pharmaceutical agent data 203 by a pharmaceutical agent ID 204. The test data 107 is data for which drug efficacy is inferred with use of the learning model 109, and has the same format as the format of the learning data 105 which includes the specimen data 200 and the pharmaceutical agent data 203. Details of the learning data 105 and the test data 107 are described later. The inference data 108 is a drug efficacy score that is inferred by the learning model 109 from the test data 107.

The learning model 109 is a deep learning model for inferring drug efficacy from data of pharmaceutical agents and data of specimens, and is a supervised learning model trained with the learning data 105, which is associated with the ground truth data 106.

The output unit 110 is an interface for outputting settings required to execute a program and a result of executing the program in a format visually recognizable to a user, and is recommended to be configured from, for example, a liquid crystal display. The input unit 111 is an interface for receiving input from an operator, and is recommended to be configured from, for example, a mouse and a keyboard. A touch panel may double as the output unit 110 and the input unit 111. Alternatively, a user terminal coupled to the information processing device 100 via a network may provide the output unit 110 and the input unit 111. In this case, the information processing device 100 may have functions of a Web server and the user terminal may access the information processing device 100 via a predetermined protocol (for example, HTTP). Further, one or both of the output unit 110 and the input unit 111 may be coupled to another information processing device so that a calculation result is output to the another information processing device and/or data required for calculation is received from the another information processing device.

The information processing device 100 may include a network interface device (not shown) that controls communication to and from another device by following a predetermined protocol.

A program executed by the processor 101 is provided via a removable medium (a CD-ROM, a flash memory, or the like) or the network to the information processing device 100, and is stored in the storage unit 104 which is a non-transitory storage medium. It is therefore recommended that the information processing device 100 include an interface through which data is read out of a removable medium.

The information processing device 100 is a computer system configured on a single physical computer or on a plurality of logically or physically configured computers, and may operate on a virtual machine built on a plurality of physical computer resources. For example, a plurality of programs which implement functions of the information processing device 100 may operate on separate physical or logical computers, or may be broken into combinations of a plurality of sub-programs so that each of the combinations operates on a single physical or logical computer.

FIG. 2A is a table for showing a configuration example of the specimen data 200 that is included in the learning data 105 as well as the test data 107 in the first embodiment.

The specimen data 200 included in the learning data 105 and the test data 107 is information about feature amounts related to specimens, and include records each of which associates a specimen ID 201 with a feature amount 202.

FIG. 2B is a table for showing a configuration example of the pharmaceutical agent data 203 that is included in the learning data 105 as well as the test data 107 in the first embodiment.

The pharmaceutical agent data 203 included in the learning data 105 and the test data 107 is information about feature amounts related to pharmaceutical agents, and include a pharmaceutical agent ID 204 and a feature amount 205. Each pharmaceutical agent ID 204 is associated with the feature amount 205.

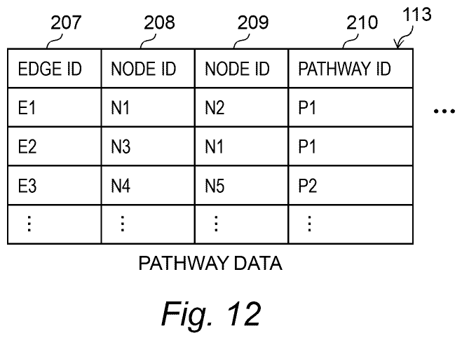

A pathway, for example, is usable for the feature amount 202 of the specimen data 200 and the feature amount 205 of the pharmaceutical agent data 203. A pathway is, as shown in FIG. 12 referred to a third embodiment of this invention, data including one or more edges which couple one node to another node, and, in the case of the specimen data 200 and the pharmaceutical agent data 203, is expressed as a set of edges in a graph in which a protein or a gene is a node and the degree of abnormality that occurs in the node is an edge. As a value of the feature amount 202 of the specimen data 200, expression information of a gene analyzed with use of Gene Set Enrichment Analysis (GSEA), for example, is usable. In the case of using a pathway for the feature amount 202 of the specimen data 200, the feature amount 202 indicates, for example, the degree of abnormality of the specimen with respect to a predetermined protein or gene in a predetermined pathway. Specifically, when a predetermined specimen is used, the value of the feature amount 202 is high in a case in which the degree of abnormality that occurs in a protein or a gene on a pathway is high, and the value of the feature amount 202 is low in a case in which the degree of abnormality that occurs in a protein or a gene on a pathway is low. A value of the feature amount 205 of the pharmaceutical agent data 203 indicates, for example, treatment effectiveness. Specifically, when a predetermined pharmaceutical agent is used, the value of the feature amount 205 is high in a case of a pathway including a gene that generates a functional-gene product (for example, a protein) acting to increase treatment effectiveness, and the value of the feature amount 205 is low in a case of a pathway including a large number of genes that generate a functional-gene product (for example, a protein) acting to decrease treatment effectiveness.

The format of the learning data and the test data is not limited to FIG. 2A and FIG. 2B. For example, the learning data and the test data may be a single piece of table data in which pharmaceutical agent data and specimen data are linked to each other by some method.

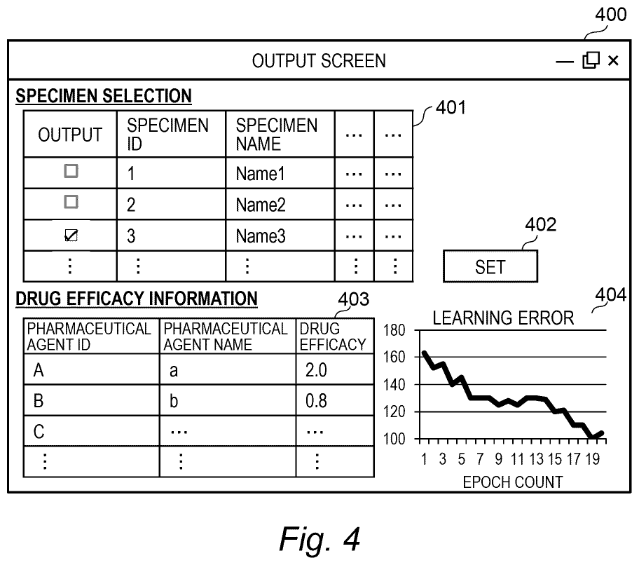

FIG. 3 is a diagram for illustrating an example of a setting screen 300 output by the information processing device 100 of the first embodiment.

The setting screen 300 is displayed on the output unit 110, and is used to set the ground truth data 106 and the learning data 105 which are input data for training the learning model 109, set the test data 107 for inferring drug efficacy with use of the trained learning model 109, and set parameters to be used in learning and inference.

The setting screen 300 includes a learning mode button 301, a learning data file input field 302, a ground truth data file input field 303, a test mode button 304, a learning model file input field 314, a test data file input field 305, a set button 306, a settings file input field 307, an edit button 308, and a set button 309.

The user can operate the learning mode button 301 to switch to a learning mode, specify, out of files stored in the storage unit 104, a file of the learning data 105 (the specimen data 200 and the pharmaceutical agent data 203) and a file of the ground truth data 106 that are to be used for training the learning model 109 in the learning data file input field 302 and the ground truth data file input field 303, respectively, and operate the set button 306 to input the specified files to the memory 103.

The user can also operate the test mode button 304 to switch to a test mode, specify, out of the files stored in the storage unit 104, a file of the trained learning model 109 and the test data 107 (the specimen data 200 and the pharmaceutical agent data 203) for evaluating performance of the learning model 109 in the learning model file input field 314 and the test data file input field 305, respectively, and operate the set button 306 to input the specified files to the memory 103.

The user can also specify a settings file that specifies a condition for inferring drug efficacy with the use of the learning model 109 set in the learning model file input field 314, and operate the set button 309 to input the specified file to the memory 103. The user can also operate the edit button 308 to activate a settings file editor and edit contents of the settings file (for example, an internal parameter of the model, an epoch count k, and hyperparameters (a learning rate and a batch size)) with the settings file editor. Parameters that have been optimized in learning executed in advance may also be set.

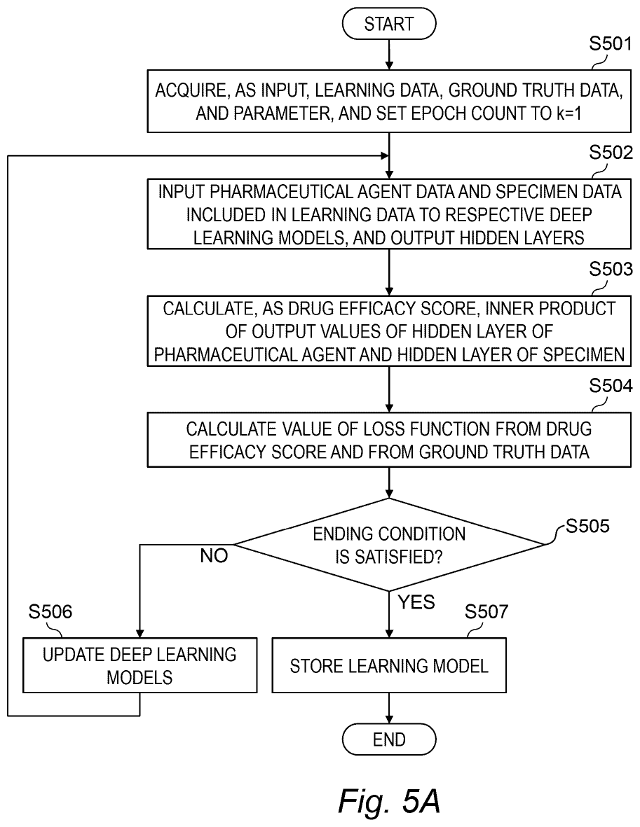

FIG. 4 is a diagram for illustrating an example of an output screen 400 output by the information processing device 100 of the first embodiment.

The output screen 400 is displayed on the output unit 110, and includes a specimen selection area 401, a set button 402, and a drug efficacy information display area 403. The output screen 400 may include a learning error display area 404 as well.

The specimen selection area 401 includes fields for a specimen ID, a specimen name, and selecting output, and is displayed in a table format. The drug efficacy information display area 403 includes a pharmaceutical agent ID, a pharmaceutical agent name, and a drug efficacy inference result, and is displayed in a table format. When the user selects a specimen in the specimen selection area 401 and operates the set button 402, the processor 101 uses the learning model 109 to infer drug efficacy, and outputs drug efficacy information of the selected specimen in the drug efficacy information display area 403. In the drug efficacy information display area 403, pharmaceutical agents are displayed in order of contribution to inference. Display of pharmaceutical agents in the drug efficacy information display area 403 enables the user to check drug efficacy inferred with respect to the selected specimen, pharmaceutical agent IDs, and pharmaceutical agent names. The learning error display area 404 displays transitions of a loss function in relation to the epoch count.

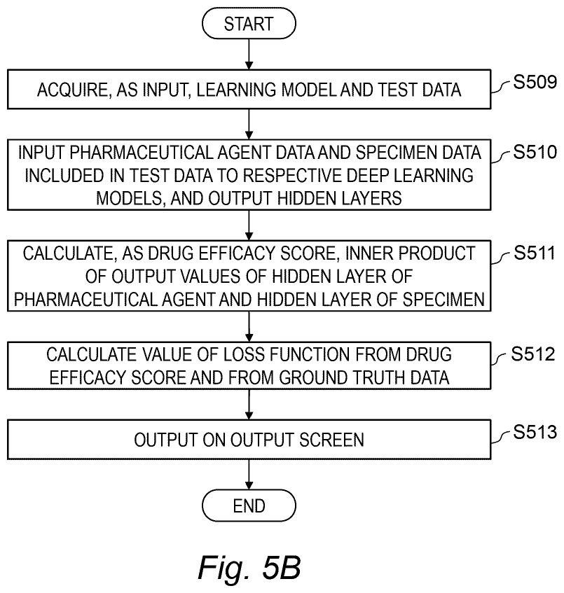

FIG. 5A is a flow chart of learning processing that is executed by the information processing device 100 of the first embodiment. In the learning processing, the learning data 105 (the specimen data 200 and the pharmaceutical agent data 203) and the ground truth data 106 are used to generate the learning model 109.

In Step S501, the processor 101 receives input of the learning data 105 and the ground truth data 106, and settings of an internal parameter which are specified by the user on the setting screen 300. The processor 101 also sets the epoch count k to 1.

In Step S502, the processor 101 inputs the specimen data 200 and the pharmaceutical agent data 203 that are included in the learning data 105 to the learning model 109 so that values of hidden layers are output.

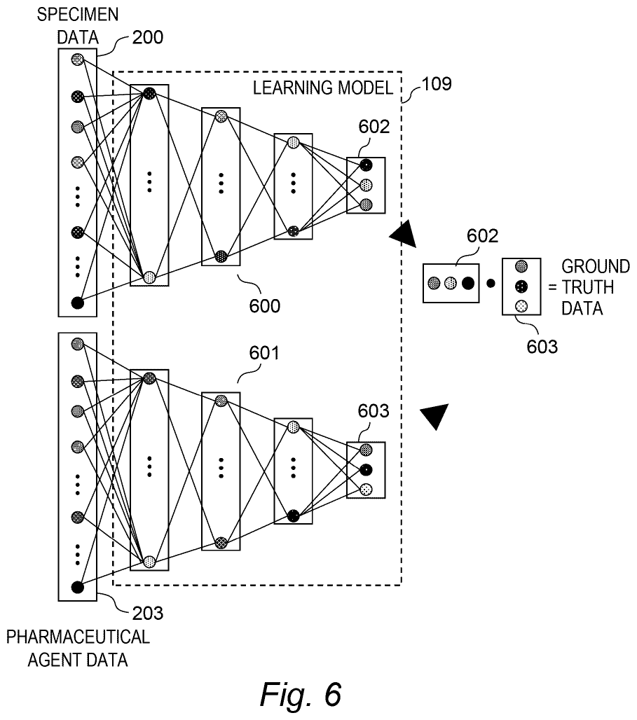

In Step S503, the processor 101 calculates an inner product of an output value 602 of the hidden layer of the specimens and an output value 603 of the hidden layer of the pharmaceutical agents as a drug efficacy score. As illustrated in FIG. 6, the learning model 109 includes a deep learning model 600 to which the specimen data 200 is input and a deep learning model 601 to which the pharmaceutical agent data 203 is input, and calculates an inner product of the output value 602 of a hidden layer of the deep learning model 600 and the output value 603 of a hidden layer of the deep learning model 601. The hidden layer of the deep learning model 600 may indicate the degree of abnormality of a gene in a specimen, and the hidden layer of the deep learning model 601 may indicate the degree of treatment effectiveness of a pharmaceutical agent. The deep learning model 600 and the deep learning model 601 are equal to each other in the number of layers from an input layer to the hidden layer. Output of the hidden layer of the deep learning model 600 and output of the hidden layer of the deep learning model 601 are equal to each other in the number of dimensions. The degree of similarity between a plurality of phenomena is calculable as an inner product of output values of hidden layers by equalizing the output values of the hidden layers in the number of dimensions. In the deep learning models 600 and 601, calculation of drug efficacy as an inner product of output values of hidden layers enables analysis of a relationship between predetermined related information such as a pathway and a plurality of phenomena, even in a case of a plurality of pieces of data different from one another in data type.

In Step S504, the processor 101 calculates a value of the loss function based on the drug efficacy score and the ground truth data 106.

In Step S505, the processor 101 determines whether a condition for ending learning is satisfied. For example, in a case in which the value of the loss function calculated in Step S504 by comparing the drug efficacy score and the ground truth data 106 is equal to or less than a threshold value a predetermined number of times in succession, it means that a desired learning model has been obtained, and this is a recommended time to end learning. Another recommended time to end learning is when the epoch count k is equal to or more than a predetermined value determined as a condition for ending learning.

In Step S506, when the condition for ending learning is unsatisfied, the processor 101 updates the deep learning models 600 and 601 so that the value of the loss function calculated in Step S504 is minimized, and adds 1 to the epoch count k.

The process then returns to Step S502 to move on to processing of the next record of the learning data 105.

In Step S507, when the condition for ending learning is satisfied, the processor 101 stores the learning model 109 including the updated deep learning models 600 and 601 in the storage unit 104.

FIG. 5B is a flow chart of test processing that is executed by the information processing device 100 of the first embodiment. In the test processing, the test data 107 (the specimen data 200 and the pharmaceutical agent data 203) and the ground truth data 106 are used to verify performance of the generated learning model 109.

In Step S509, the processor 101 receives input of the learning model 109, the test data 107 of a specimen, and the ground truth data 106, and settings of an internal parameter which are selected by the user on the output screen 400.

In Step S510, the processor 101 inputs the specimen data 200 and the pharmaceutical agent data 203 that are included in the test data 107 to the learning model 109 so that values of hidden layers are output.

In Step S511, the processor 101 calculates an inner product of the output value 602 of the hidden layer of the specimens and the output value 603 of the hidden layer of the pharmaceutical agents as a drug efficacy score.

In Step S512, the processor 101 calculates a value of a loss function based on the drug efficacy score and the ground truth data 106.

In Step S513, the processor 101 outputs display data for displaying drug efficacy for each pharmaceutical agent and learning errors in the form of a loss function on the output unit 110.

FIG. 6 is a diagram for illustrating the learning model 109 in the first embodiment.

As described above, the learning model 109 includes the deep learning model 600 to which the specimen data 200 is input and the deep learning model 601 to which the pharmaceutical agent data 203 is input. With the specimen data 200 input to the deep learning model 600 and the pharmaceutical agent data 203 input to the deep learning model 601, the learning model 109 is trained so that the inner product of the output values of the hidden layers matches the ground truth data 106.

The output value 602 of the hidden layer of the specimens which is obtained by inputting the specimen data 200 to the deep learning model 600 indicates the degree of abnormality of a gene at each bit of the hidden layer. The output value 603 of the hidden layer of pharmaceutical agents which is obtained by inputting the pharmaceutical agent data 203 to the deep learning model 601 indicates the degree of treatment effectiveness of a pharmaceutical agent at each bit of the hidden layer.

The number of layers of the deep learning model 600 and the number of layers of the deep learning model 601 are equal to each other, and the number of dimensions of the deep learning model 600 and the number of dimensions of the deep learning model 601 are equal to each other.

Thus, training is executed so that the drug efficacy score that is the inner product of the output values of the hidden layers of the two deep learning models 600 and 601 approaches the ground truth data 106, and, accordingly, a feature amount that is highly related to abnormality and highly related to treatment effectiveness of a pharmaceutical agent has a high drug efficacy score.

As described above, the information processing device 100 of the first embodiment includes an arithmetic device (the processor 101) that executes predetermined processing, the input unit 111 that receives, as input, pieces of related information (the specimen data 200 and the pharmaceutical agent data 203) pertaining respectively to a plurality of phenomena, the storage unit 104 that stores the learning model 109 which derives an inference result of the pieces of related information pertaining to the phenomena, and the output unit 110 that outputs the inference result. The arithmetic device calculates an inner product of the first output value 602 of a hidden layer of the learning model 109 to which a first phenomenon (the specimen data 200) out of the plurality of phenomena is input and the second output value 603 of a hidden layer of the learning model 109 to which a second phenomenon (the pharmaceutical agent data 203) out of the plurality of phenomena is input, and executes deep learning of the learning model based on the pieces of related information pertaining to the phenomena in a manner that decreases, for each combination of phenomena (a specimen and a pharmaceutical agent), a difference between the calculated inner product and the ground truth data 106 out of the plurality of phenomena. Accordingly, the information processing device 100 of the first embodiment can provide a drug efficacy inference model that is highly reliable, and enables the user to check, for each piece of specimen data 200, inferred drug efficacy and pharmaceutical agents.

With the related art, based on results of trials that use published data, output of a final layer of a model in each pair of a specimen and a pharmaceutical agent is subjected to dimensionality reduction to be reduced to two dimensions by tSNE. A latent space is thus visualized for observation of a cluster configured for each pharmaceutical agent, and overfitting with respect to a difference from one pharmaceutical agent to another pharmaceutical agent is found out through the observation. In other words, a difference from one pharmaceutical agent to another pharmaceutical agent significantly contributes to prediction of effectiveness, and it means that the related art has been unsuccessful in providing prediction that takes a relationship between an expected inhibition target of a pharmaceutical agent and abnormality of a specimen into account. In prediction by the learning model 109 in the first embodiment, on the other hand, data about specimens and data about pharmaceutical agents are independently learned in a manner that decreases the difference between an inner product of output values of the final layer and the ground truth data 106. Accordingly, the first embodiment simultaneously accomplishes prevention of overfitting with respect to differentiation between pharmaceutical agents and between specimens and generation of the learning model 109 in which a prediction mechanism for predicting drug efficacy expected to be possessed by a pharmaceutical agent in inhibition of abnormality of a specimen is incorporated.

Although generation of a learning model is described in the first embodiment, specimen data and pharmaceutical agent data may be newly received as input in the input unit 111 so that the processor 101 uses a learning model that has learned the specimen data and the pharmaceutical agent data to infer treatment effectiveness expected when a pharmaceutical agent of the pharmaceutical agent data is used on a specimen of the specimen data. This enables precise calculation of treatment effectiveness of a pharmaceutical agent of the pharmaceutical agent data on a specimen of the specimen data. The calculated treatment effectiveness may be output on the output unit 110.

### Second Embodiment

In a second embodiment of this invention, the device described in the first embodiment calculates, for each feature amount of the pharmaceutical agent data 203 and the specimen data 200, the degree of contribution to prediction, to thereby improve explainability. In the second embodiment, differences from the first embodiment described above are mainly described, and components and processing that are the same as the components and the processing in the first embodiment are denoted by the same reference symbols in order to omit descriptions thereof.

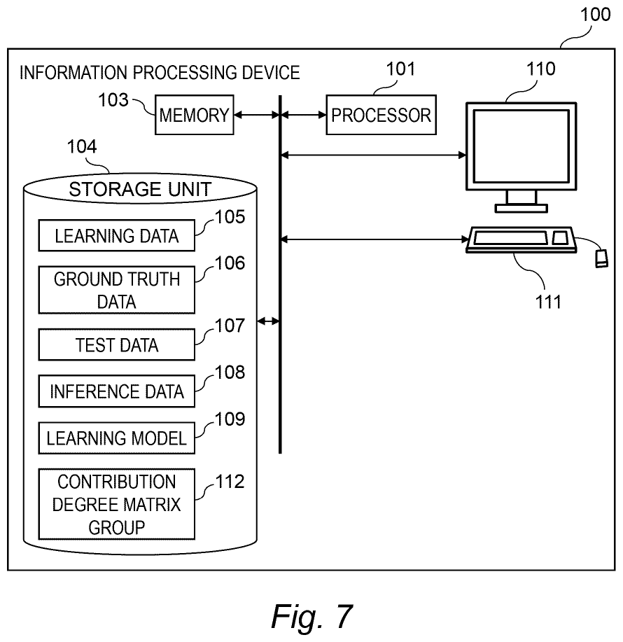

FIG. 7 is a diagram for illustrating a hardware configuration of the information processing device 100 of the second embodiment.

The information processing device 100 of the second embodiment is configured from a computer including the processor 101, the arithmetic unit 102, the memory 103, a storage unit 104, the output unit 110, and the input unit 111.

The processor 101, the arithmetic unit 102, the memory 103, the output unit 110, and the input unit 111 are the same as in the first embodiment described above.

The storage unit 104 is configured from a storage apparatus that provides a large-capacity and non-volatile storage area, for example, a magnetic storage apparatus (HDD) or a flash memory (SSD). The storage unit 104 stores the data (the learning data 105, the ground truth data 106, the test data 107, the inference data 108, the learning model 109, and a contribution degree matrix group 112) used by the processor 101 when executing the program, and the program executed by the processor 101. Specifically, the program is read out of the storage unit 104, loaded onto the memory 103, and is executed by the processor 101 to implement the functions of the information processing device 100.

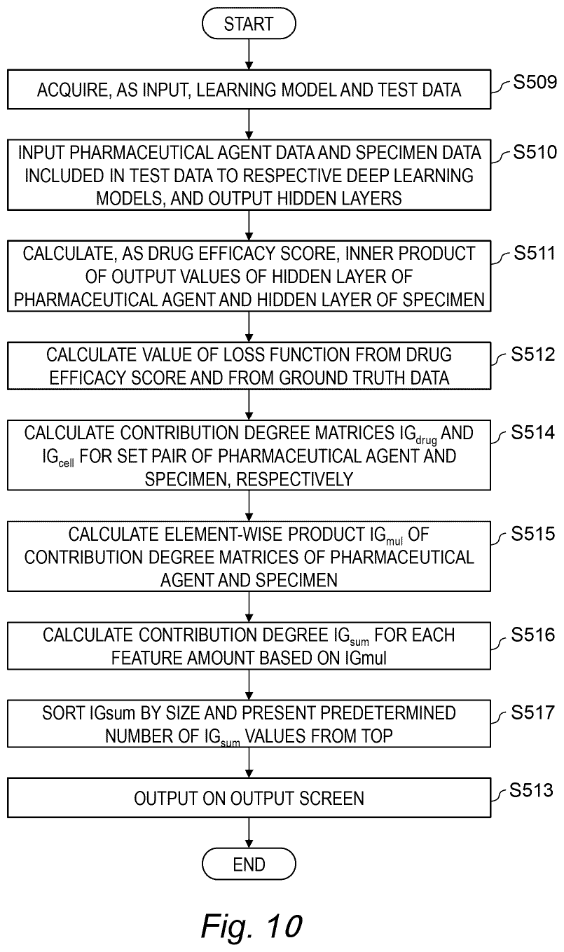

The contribution degree matrix group 112 indicates the degree of contribution of the specimen data 200 and the pharmaceutical agent data 203, and a configuration thereof is described with reference to FIG. 10.

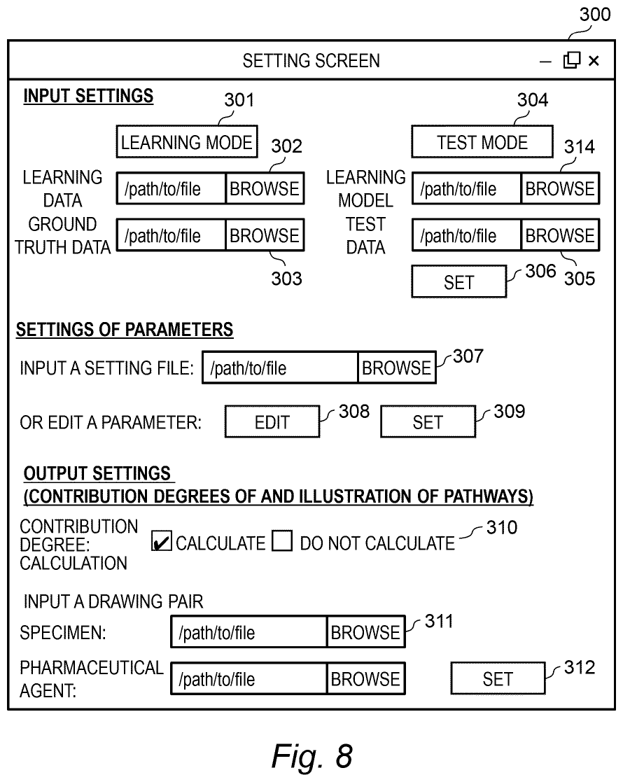

FIG. 8 is a diagram for illustrating an example of a setting screen 300 output by the information processing device 100 of the second embodiment.

The setting screen 300 in the second embodiment includes the learning mode button 301, the learning data file input field 302, the ground truth data file input field 303, the test mode button 304, the learning model file input field 314, the test data file input field 305, the set button 306, the settings file input field 307, the edit button 308, the set button 309, a contribution degree calculation selection field 310, a drawing pair input field 311, and a set button 312.

The learning mode button 301, the learning data file input field 302, the ground truth data file input field 303, the test mode button 304, the test data file input field 305, the set button 306, the settings file input field 307, the edit button 308, and the set button 309 are the same as in the first embodiment described above.

The user can select whether to calculate and output the degree of contribution in the contribution degree calculation selection field 310, specify a file indicating a pair of a pharmaceutical agent and a specimen of which the degree of contribution is to be output in the drawing pair input field 311, and operate the set button 312 to input the specified file to the memory 103 and display the degree of contribution on the output screen 400. An input field for setting the number of feature amounts to be displayed as feature amounts that contribute greatly to prediction may also be provided.

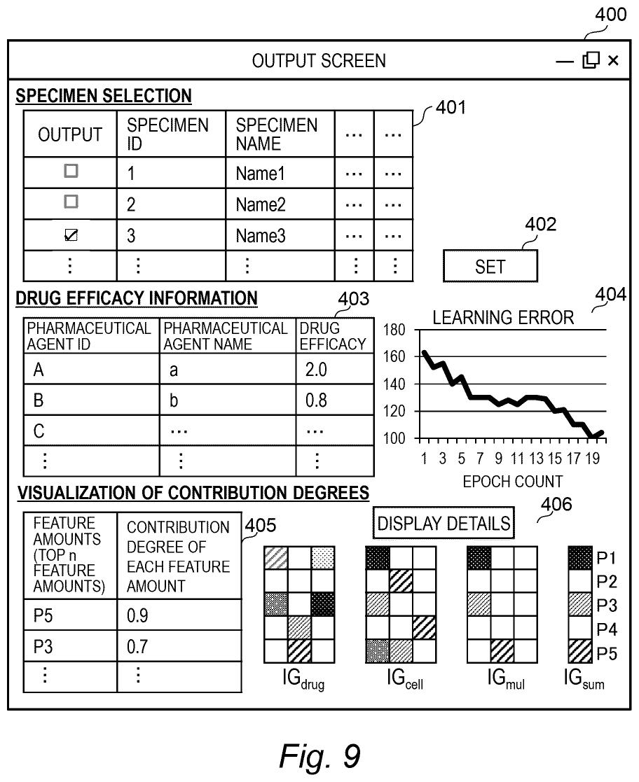

FIG. 9 is a diagram for illustrating an example of the output screen 400 output by the information processing device 100 of the second embodiment.

The output screen 400 in the second embodiment is displayed on the output unit 110, and includes the specimen selection area 401, the set button 402, the drug efficacy information display area 403, the learning error display area 404, a contribution degree visualization area 405, and a details display area 406.

The specimen selection area 401, the set button 402, the drug efficacy information display area 403, and the learning error display area 404 are the same as in the first embodiment described above.

In the contribution degree visualization area 405, a feature amount large in the degree of contribution to drug efficacy for each of the pharmaceutical agent data 203 and the specimen data 200, and the degree of contribution of the feature amount are output. When the user operates a display details button, four diagrams are further output in the details display area 406. Of the diagrams, IG_{drug} is an illustration of the degree of contribution of a feature amount of a pharmaceutical agent to drug efficacy, IG_{cell} is an illustration of the degree of contribution of a feature amount of a specimen to drug efficacy, IGₘᵤₗ is an illustration of an element-wise product of IG_{drug} and IG_{cell}, and IGₛᵤₘ is an illustration of a sum of IGₘᵤₗ in a dimension direction. In IG_{drug}, IG_{cell}, and IGₘᵤₗ, feature amounts (P1 to P5) are represented in a longitudinal direction, and neurons of the hidden layer are represented in a lateral direction.

Learning processing of the information processing device 100 of the second embodiment is the same as in the first embodiment described above.

FIG. 10 is a flow chart of test processing that is executed by the information processing device 100 of the second embodiment.

Step S509 to Step S512 of the test processing are the same as in the first embodiment described above.

In Step S514, the processor 101 calculates integrated gradients to calculate the degrees of contribution of neurons of the hidden layers to drug efficacy. For example, a contribution degree matrix IG_{drug} of the pharmaceutical agent data 203 is calculated with use of Equation 1. In Equation 1, i is a suffix related to feature amounts, j is a suffix of neurons in the final layer of hidden layers, x represents a feature amount vector, x′ represents a base line, F(x) represents output of the final layer of hidden layers, and m represents an integer. A contribution degree matrix IG_{cell} of the specimen data 200 is similarly calculated with the use of Equation 1. An effect on drug efficacy with regards to the pharmaceutical agent data 203 is thus calculable as the degree of contribution.

$${IG}_{drugij} = ( {x}_{i} - {x}_{i}^{′} ) {\sum }_{k = 1}^{m} \frac{\partial {F}_{j} ( \vec{{x}^{′}} + \frac{k}{m} \times ( \vec{x} - \vec{{x}^{′}} ) )}{\partial {x}_{i}} \times \frac{1}{m} \qquad [ Equation 1 ]$$

The contribution degree matrix IG_{cell} of the specimen data 200 is regarded as a similar effect, and an effect on drug efficacy in the specimen data 200 is thus calculable.

$${IG}_{cellij} = ( {x}_{i} - {x}_{i}^{′} ) {\sum }_{k = 1}^{m} \frac{\partial {F}_{j} ( \vec{{x}^{′}} + \frac{k}{m} \times ( \vec{x} - \vec{{x}^{′}} ) )}{\partial {x}_{i}} \times \frac{1}{m} \qquad [ Equation 2 ]$$

In S515, the processor 101 uses Equation 3 to calculate an element-wise product IGₘᵤₗ of the contribution degree matrices of the pharmaceutical agent data 203 and the specimen data 200.

$${IG}_{m u l i j} = {IG}_{d r u g i j} \cdot {IG}_{c e l l i j} \qquad [ Equation 3 ]$$

In S516, the processor 101 uses Equation 4 to calculate, from IGₘᵤₗ, a contribution degree IGₛᵤₘ for each feature amount.

$${IG}_{s u m i} = {\sum }_{j} {IG}_{mulij} \qquad [ Equation 4 ]$$

In S517, the processor 101 sorts IGₛᵤₘ by size, and outputs a predetermined number of contribution degrees, counted from a first place in the sorted order.

In the equations given above, integrated gradients of the pharmaceutical agent data 203 satisfy Equation 5.

$$( \vec{x} ) - {F}_{j} ( \vec{{x}^{′}} ) = {\sum }_{i} {IG}_{drugij} \qquad [ Equation 5 ]$$

Integrated gradients of the specimen data 200 similarly satisfy Equation 6.

$${F}_{j} ( \vec{x} ) - {F}_{j} ( \vec{{x}^{′}} ) = {\sum }_{i} {IG}_{cellij} \qquad [ Equation 6 ]$$

A relationship between the contribution degree IGₛᵤₘ and the drug efficacy score of each feature amount is expressed by Equation 7 based on a relationship of Equations 5 and 6. A feature amount larger in the contribution degree IGₛᵤₘ can be considered to be a feature amount that contributes more to improvement of drug efficacy.

$$drug score = {\sum }_{i} {IG}_{s u m i} \qquad [ Equation 7 ]$$

In S513, the processor 101 outputs feature amounts large in the degree of contribution and the degrees of contribution of the feature amounts on the output screen 400.

As described above, the information processing device 100 of the second embodiment of this invention calculates the degree of contribution of each feature amount. The information processing device 100 of the second embodiment accordingly enables the user to find out actions of gene abnormality and of a pharmaceutical agent that contribute to treatment, and can enhance reliability of a drug efficacy inference model in clinical application.

### Third Embodiment

In the third embodiment, for actual utilization in clinical application, pathways which are feature amounts that greatly contribute to prediction are illustrated in order to support decision making. In the third embodiment, differences from the first and second embodiments described above are mainly described, and components and processing that are the same as the components and the processing in the first and second embodiments are denoted by the same reference symbols in order to omit descriptions thereof.

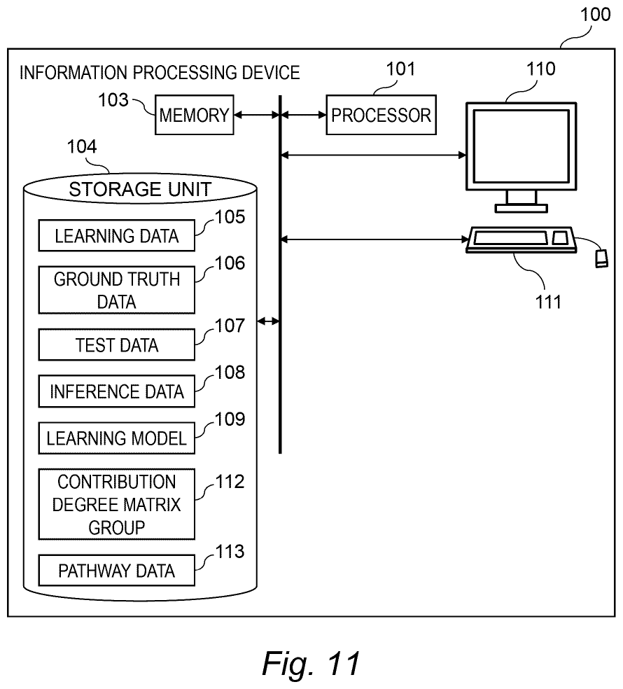

FIG. 11 is a diagram for illustrating a hardware configuration of the information processing device 100 of the third embodiment.

The information processing device 100 of the third embodiment is configured from a computer including the processor 101, the arithmetic unit 102, the memory 103, a storage unit 104, the output unit 110, and the input unit 111.

The processor 101, the arithmetic unit 102, the memory 103, the output unit 110, and the input unit 111 are the same as in the first embodiment described above.

The storage unit 104 is configured from a storage apparatus that provides a large-capacity and non-volatile storage area, for example, a magnetic storage apparatus (HDD) or a flash memory (SSD). The storage unit 104 stores the data (the learning data 105, the ground truth data 106, the test data 107, the inference data 108, the learning model 109, the contribution degree matrix group 112, and pathway data 113) used by the processor 101 when executing the program, and the program executed by the processor 101. Specifically, the program is read out of the storage unit 104, loaded onto the memory 103, and is executed by the processor 101 to implement the functions of the information processing device 100.

A pathway defined by the pathway data 113 is configured from one or a plurality of nodes coupling one edge to another edge. A configuration example of the pathway data 113 is described with reference to FIG. 12.

FIG. 12 is a table for showing a configuration example of the pathway data 113 in the third embodiment.

The pathway data 113 includes an edge ID 207, a node ID 208, a node ID 209, and a pathway ID 210. The edge ID 207 is identification information of an edge. The node IDs 208 and 209 are pieces of identification information of nodes located at ends of the edge. The pathway ID 210 is identification information of a pathway including the edge.

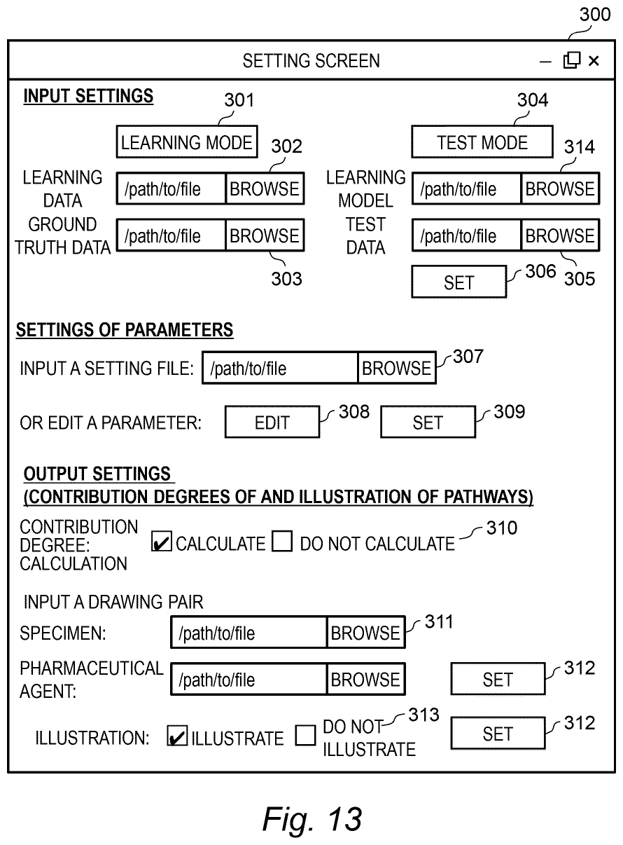

FIG. 13 is a diagram for illustrating an example of a setting screen 300 output by the information processing device 100 of the third embodiment.

The setting screen 300 in the third embodiment includes the learning mode button 301, the learning data file input field 302, the ground truth data file input field 303, the test mode button 304, the learning model file input field 314, the test data file input field 305, the set button 306, the settings file input field 307, the edit button 308, the set button 309, the contribution degree calculation selection field 310, the drawing pair input field 311, the set button 312, and an illustration selection field 313.

The learning mode button 301, the learning data file input field 302, the ground truth data file input field 303, the test mode button 304, the learning model file input field 314, the test data file input field 305, the set button 306, the settings file input field 307, the edit button 308, the set button 309, the contribution degree calculation selection field 310, the drawing pair input field 311, and the set button 312 are the same as in the first and second embodiments described above.

The user can select whether to calculate and output the degree of contribution in the contribution degree calculation selection field 310, specify a file indicating a pair of a pharmaceutical agent and a specimen of which the degree of contribution is to be output in the drawing pair input field 311, select whether to illustrate a pathway in the illustration selection field 313, and operate the set button 312 to input the specified file to the memory 103 and display the degree of contribution on the output screen 400. An input field for setting the number of feature amounts to be displayed as feature amounts that contribute greatly to prediction may also be provided.

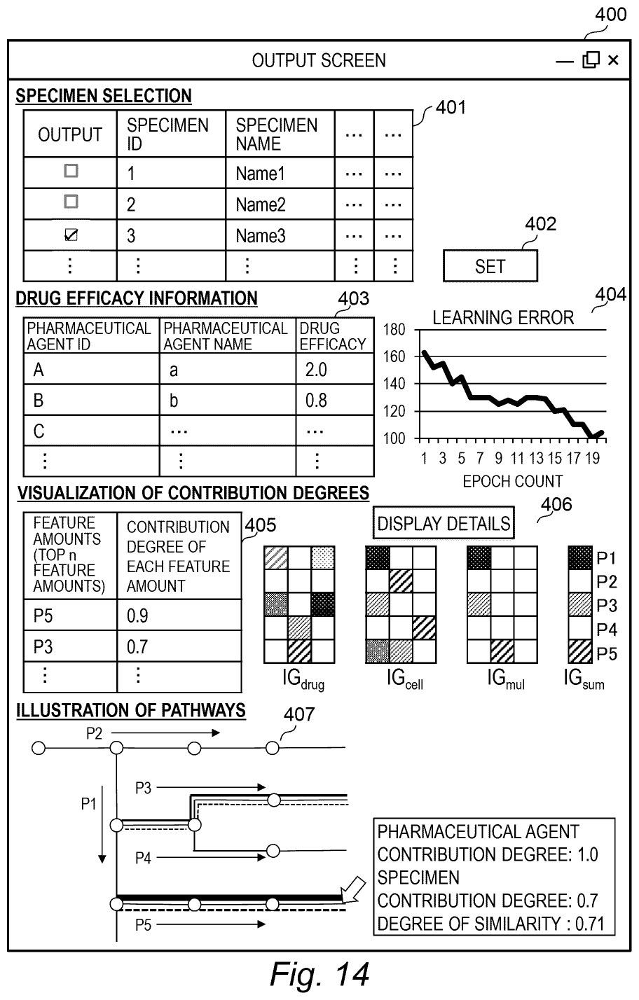

FIG. 14 is a diagram for illustrating an example of an output screen 400 output by the information processing device 100 of the third embodiment.

The output screen 400 in the third embodiment is displayed on the output unit 110, and includes the specimen selection area 401, the set button 402, the drug efficacy information display area 403, the learning error display area 404, the contribution degree visualization area 405, the details display area 406, and a pathway display area 407.

The specimen selection area 401, the set button 402, the drug efficacy information display area 403, the learning error display area 404, the contribution degree visualization area 405, and the details display area 406 are the same as in the first and second embodiments described above.

In the pathway display area 407, pathways which are feature amounts that contribute greatly to prediction are illustrated. In the illustrated example, a solid line displayed above an edge is a pathway (feature amount) that has contributed to the drug efficacy score in the pharmaceutical agent data 203, and a dotted line displayed below the edge is a pathway (feature amount) that has contributed to the drug efficacy score in the specimen data 200. Thicknesses of the solid line and the dotted line indicate how large the degrees of contribution are. The mode of displaying pathways that contribute greatly is not limited to the mode described above.

Learning processing of the information processing device 100 of the third embodiment is the same as in the first embodiment described above.

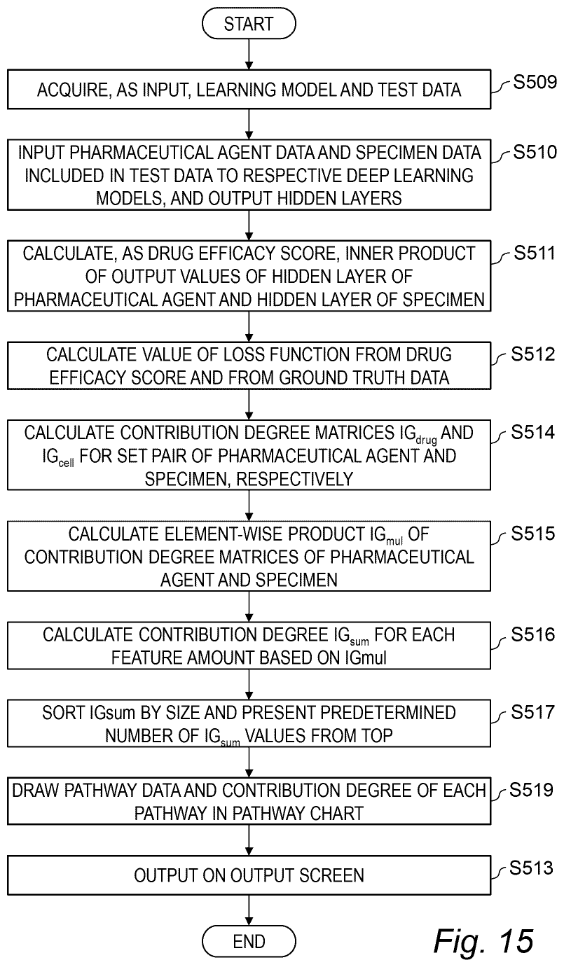

FIG. 15 is a flow chart of test processing that is executed by the information processing device 100 of the third embodiment.

Step S509 to Step S517 of the test processing are the same as in the first and second embodiments described above.

In Step S519, the processor 101 draws a pathway chart based on the pathway data 113 and the degree of contribution of each pathway.

As described above, according to the information processing device 100 of the third embodiment of this invention, feature amounts (pathways) that greatly contribute to prediction are illustrated, and the user can thus understand and compare the degrees of contribution intuitively. This enables the user to make a rational and effective decision in a clinical setting.

The descriptions given above are about examples in which feature amounts are pathways with specimens and pharmaceutical agents set as phenomena, and a target of inference is drug efficacy. However, this invention is also applicable to other feature amounts and other targets of inference. In addition, although the input data is specimen data and pharmaceutical agent data in the examples described above, this invention is also applicable to other types of input data. For example, the input data may be fewness of roads and the number of accidents, feature amounts may be roads, and the target of inference may be the degree of traffic jam. The number of pieces of input data may be three or more instead of two. Three pieces of input data may be, for example, pharmaceutical agents and specimens in the embodiments described above, and the degree of relevance to disorder on each pathway.

This invention is not limited to the above-described embodiments but includes various modifications. The above-described embodiments are explained in details for better understanding of this invention and are not limited to those including all the configurations described above. A part of the configuration of one embodiment may be replaced with that of another embodiment; the configuration of one embodiment may be incorporated to the configuration of another embodiment. A part of the configuration of each embodiment may be added, deleted, or replaced by that of a different configuration.

The above-described configurations, functions, processing modules, and processing means, for all or a part of them, may be implemented by hardware: for example, by designing an integrated circuit, and may be implemented by software, which means that a processor interprets and executes programs providing the functions.

The information of programs, tables, and files to implement the functions may be stored in a storage device such as a memory, a hard disk drive, or an SSD (a Solid State Drive), or a storage medium such as an IC card, or an SD card.

The drawings illustrate control lines and information lines as considered necessary for explanation but do not illustrate all control lines or information lines in the products. It can be considered that almost of all components are actually interconnected.

What is claimed is:

1. An information processing device, comprising:
an arithmetic device configured to execute predetermined processing:
an input unit configured to receive, as input, pieces of related information pertaining respectively to a plurality of phenomena;
a storage unit configured to store a learning model which derives an inference result of the pieces of related information pertaining to the plurality of phenomena; and
an output unit configured to output the inference result,
wherein the arithmetic device is configured to:
calculate an inner product of a first output value of a hidden layer of the learning model to which a first phenomenon out of the plurality of phenomena is input and a second output value of a hidden layer of the learning model to which a second phenomenon out of the plurality of phenomena is input; and
execute deep learning of the learning model based on the pieces of related information pertaining to the plurality of phenomena in a manner that decreases, for each combination of phenomena, a difference between the calculated inner product and ground truth data out of the plurality of phenomena.

2. The information processing device according to claim 1,
wherein, in the learning model, the number of layers from an input layer to a hidden layer of the first phenomenon and the number of layers from an input layer to a hidden layer of the second phenomenon are equal to each other, and
wherein an output value of the hidden layer of the first phenomenon and an output value of the hidden layer of the second phenomenon have the same number of dimensions.

3. The information processing device according to claim 1,
wherein the first phenomenon is a specimen,
wherein the second phenomenon is a pharmaceutical agent, and
wherein the inference result is treatment effectiveness of the pharmaceutical agent on the specimen.

4. The information processing device according to claim 3,
wherein a hidden layer of the first phenomenon indicates a degree of abnormality of a gene in the specimen, and
wherein a hidden layer of the second phenomenon indicates a degree of treatment effectiveness of the pharmaceutical agent.

5. The information processing device according to claim 3,
wherein the input unit is configured to receive, as input, specimen-related information pertaining to a specimen and pharmaceutical agent-related information pertaining to effectiveness of a pharmaceutical agent, and
wherein the arithmetic device is configured to:
calculate an inner product of the first output value of the hidden layer of the learning model to which the specimen-related information is input and the second output value of the hidden layer of the learning model to which the pharmaceutical agent-related information is input; and
execute deep learning of the learning model based on the specimen-related information and the pharmaceutical agent-related information in a manner that decreases, for each combination of the specimen and the pharmaceutical agent, a difference between the calculated inner product and the ground truth data.

6. The information processing device according to claim 1,
wherein the pieces of related information pertaining to the plurality of phenomena are expressed by the same feature amount.

7. The information processing device according to claim 5,
wherein the arithmetic device is configured to calculate, by using integrated gradients with respect to the hidden layers, for each of a plurality of matters, a degree of contribution to the inference result, and
wherein the output unit is configured to output the calculated degree of contribution.

8. The information processing device according to claim 7,
wherein the arithmetic device is configured to calculate a degree of contribution of the specimen-related information and the pharmaceutical agent-related information to the inference result based on an element-wise product of the calculated degrees of contribution of the plurality of matters, and
wherein the output unit is configured to output the calculated degree of contribution.

9. The information processing device according to claim 7,
wherein the pieces of related information are values corresponding to pathways that are expressed by graphs in which a protein and a gene are nodes, and a degree of abnormality that occurs in each of the nodes is an edge,
wherein the arithmetic device is configured to calculate, for each of the pathways, a degree of contribution to the inference result, and
wherein the output unit is configured to output the calculated degree of contribution along with the each of the pathways.

10. The information processing device according to claim 5,
wherein the input unit is configured to receive pharmaceutical agent data and specimen data as input,
wherein the arithmetic device is configured to calculate treatment effectiveness of a pharmaceutical agent of the pharmaceutical agent data on a specimen of the specimen data based on the pharmaceutical agent data and the specimen data which are received by the input unit, and on the learning model, and
wherein the output unit is configured to output the treatment effectiveness.

11. An information processing method, which is executed by an information processing device,
the information processing device including: an arithmetic device configured to execute predetermined processing; an input unit configured to receive, as input, pieces of related information pertaining respectively to a plurality of phenomena; a storage unit configured to store a learning model which derives an inference result of the pieces of related information pertaining to the plurality of phenomena; and an output unit configured to output the inference result,
the information processing method comprising:
calculating, by the arithmetic device, an inner product of a first output value of a hidden layer of the learning model to which a first phenomenon out of the plurality of phenomena is input and a second output value of a hidden layer of the learning model to which a second phenomenon out of the plurality of phenomena is input; and
executing, by the arithmetic device, deep learning of the learning model based on the pieces of related information pertaining to the plurality of phenomena in a manner that decreases, for each combination of phenomena, a difference between the calculated inner product and ground truth data out of the plurality of phenomena.

12. A non-transitory machine-readable storage medium, containing at least one sequence of instructions for allocating a plurality of virtual machines by using resources included in a plurality of physical computers,
the information processing device including: an arithmetic device configured to execute predetermined processing; an input unit configured to receive, as input, pieces of related information pertaining respectively to a plurality of phenomena; a storage unit configured to store a learning model which derives an inference result of the pieces of related information pertaining to the plurality of phenomena; and an output unit configured to output the inference result,
the instructions that, when executed, causes the resource management server to:
calculate an inner product of a first output value of a hidden layer of the learning model to which a first phenomenon out of the plurality of phenomena is input and a second output value of a hidden layer of the learning model to which a second phenomenon out of the plurality of phenomena is input; and
execute the deep learning of the learning model based on the pieces of related information pertaining to the plurality of phenomena in a manner that decreases, for each combination of phenomena, a difference between the calculated inner product and ground truth data out of the plurality of phenomena.
